(19) United States
(12) Patent Application Publication
(10) Pub. No.: US 2025/0352616 A1
(43) Pub. Date: Nov. 20, 2025

(54) ANNELID HEMOGLOBIN FOR USE IN THE TREATMENT OF FUCHS' DISEASE

(71) Applicants: HEMARINA, MORLAIX (FR); FONDATION A DE ROTHSCHILD, PARIS (FR)

(72) Inventors: Eric GABISON, Charenton le pont (FR); Ludovic PERRAUD, PARIS (FR); Benoit SOUQUET, PARIS (FR); Damien GUINDOLET, PARIS (FR); Franck ZAL, MORLAIX (FR)

(21) Appl. No.: 18/872,649

(22) PCT Filed: Jun. 9, 2023
(86) PCT No.: PCT/EP2023/065476

§ 371 Date: Apr. 10, 2025

(30) Foreign Application Priority Data
Jun. 9, 2022 (FR) FR2205545

(51) Int. Cl. A61K 38/17; A61P 27/02

(52) U.S. Cl. CPC A61K 38/1767; A61P 27/02

(57) ABSTRACT

The present invention relates to the use of at least one molecule selected from among an Annelid globin, an Annelid globin protomer and an Annelid extracellular hemoglobin for the treatment of Fuchs' disease.

The present invention relates to the use of at least one molecule selected from an Annelid globin, an Annelid globin protomer and an Annelid extracellular hemoglobin, for the treatment of Fuchs' disease.

The corneal endothelium, located at the posterior level of the cornea, plays a key role in maintaining same in a state of relative dehydration, essential for maintaining the clarity of the cornea (1). The corneal endothelium is a cell monolayer resting on the Descemet membrane, the basement membrane, secreted by endothelial cells and which thickens throughout life. The homeostasis of the extracellular matrix contained in the Descemet membrane is governed by a balance between the production and lysis of the extracellular matrix by the pair of metalloproteinases (MMPs) and the inhibitors (TIMPs)(2) thereof. In humans, the endothelial cell density is 4000 cells/mm² at birth and decreases gradually throughout life (3). Excessive loss of endothelial cells can be the result of multiple pathological mechanisms, such as infection (such as viral endothelitis), trauma (e.g. after cataract surgery) or else genetic pathologies. Beyond a certain threshold of loss of endothelial cells, a pathological condition called “endothelial insufficiency” develops, leading to an accumulation of water in the cornea stroma or cornea edema, responsible for a loss of corneal transparency and a decrease in visual acuity.

One of the leading causes of endothelial insufficiency is Fuchs' corneal endothelial dystrophy, which is the first indication for cornea transplant in the world (4). It is a multifactorial disease leading to cell abnormalities and increased cell death resulting in a gradual decrease in endothelial cell density. The physiopathology thereof involves stress of the endoplasmic reticulum with formation of aggresomes on the one hand (5), and a dysfunction of metalloproteinases on the other hand (6).

The disease is characterized in particular by the presence of drops in the most severely affected areas (in the center of the cornea). The drops correspond to an abnormal deposit of extra-cellular matrix at the Descemet membrane. Same can be observed clinically in 9 to 11% of women compared to 3.5 to 9% of men (7).

Only a small proportion of patients with Fuchs' dystrophy require a cornea transplant. Such patients report, at the initial stage, a transient visual blurring in the morning at wake-up, clinically corresponding to transient cornea edema. The cornea edema could be due to cornea hypoxia occurring at the pathological endothelium during prolonged eyelid closure. Literature data indicate endothelial dysfunction during prolonged contact lens wear resulting in transient cornea edema as a clinical consequence. The endothelial dysfunction is the consequence of prolonged cornea hypoxia (8).

Treatments for Fuchs' corneal endothelial dystrophy are currently limited; there are medical and surgical treatments.

On the one hand, some eye drops are used to reduce cornea edema. However, the variable effectiveness thereof is always limited to the early stages of the disease and is only a palliative treatment for a corneal disease.

On the other hand, the standard treatment remains a cornea transplant. Specifically, the graft is performed by a transposition of a sheet of endothelium and Descemet cells. Such technique, called DMEK (Descemet Membrane endothelial Keratoplasty) has the advantage of replacing only the affected tissue. However, such technique remains invasive and transient, since the life of the graft is estimated at about ten years in the absence of rejection.

There is thus a need for an effective treatment of Fuchs' corneal endothelial dystrophy that is easy to administer and non-invasive. Such a treatment would in particular compensate for the lack of graft and prevent a cornea transplant.

The present invention responds to such need.

Surprisingly, as described in the examples, the inventors have shown that intermittent hypoxia participates in the evolution of the pathology by aggravating the stress of the endoplasmic reticulum and by modifying the expression of factors involved in the pathophysiology of the disease such as BAG3 (BCL2-associated thanogene 3) and MMP2 (metalloproteinase type 2). Indeed, the inventors have identified on samples from patients suffering from the disease that BAG3, a chaperone protein involved in the formation of aggresomes in response to cellular stress, interacting with MMP2 and forming aggregates in pathological zones characterized by the presence of drops. Such formations result from an intracellular retention of MMP2 secondary to endoplasmic reticulum stress.

Moreover, some in vitro models of Fuchs dystrophy have been described to date, but the main drawback thereof is that same target only one aspect of the pathophysiology. Here again, surprisingly, the inventors have developed an in vitro cellular model of Fuchs dystrophy based on cobalt chloride, and a hypoxia/reoxygenation model mimicking the oxygen variations encountered in vivo. Th cellular model of Fuchs' dystrophy would combine several physiopathological characteristics of Fuchs' dystrophy into a single molecule, cobalt chloride.

The inventors have also discovered that the use of extracellular hemoglobin from Arenicola marina limits nocturnal oxygen fluctuations at the corneal endothelium.

The present invention thereby relates to the use of at least one molecule chosen from an Annelid globin, an Annelia globin protomer and an Annelida extracellular hemoglobin, for the treatment of Fuchs' corneal endothelial dystrophy.

The use according to the invention comprises at least one molecule chosen from an Annelid globin, an Annelid globin protomer and an Annelida extracellular hemoglobin.

Such molecule is an oxygen carrier. “Oxygen transporter” refers to a molecule apt to transport oxygen reversibly from the environment to the target cells, tissues or organs.

Annelid extracellular hemoglobin is present in all three classes of Annelida: Polychaetes, Oligochaetes and Achetes. Same is called extracellular hemoglobin because it is naturally not contained in a cell and can thus circulate freely in the bloodstream without chemical modification to stabilize or make same functional.

Annelid extracellular hemoglobin is a giant biopolymer with a molecular weight between 2000 and 4000 kDa, consisting of about 200 polypeptide chains between 4 and 12 different types that are generally grouped into two categories.

The first category, comprising 144 to 192 elements, groups together the so-called “functional” polypeptide chains which carry an active site such as heme, and are apt to reversibly bind oxygen; same are globin chains (eight types in total for the Arenicola marina hemoglobin: a1, a2, b1, b2, b3, c, d1 and d2), the masses of which are between 15 and 18 kDa. Same are very similar to vertebrate α and β chains.

The second category, with 36 to 42 elements, groups together polypeptide chains called “structure” or “linkers” having little or no active site but permitting the assembly of subunits called twelfths or protomers. There are two types of linkers, L1 and L2.

Each hemoglobin molecule consists of two superimposed hexagons called hexagonal bilayer and each hexagon is itself formed by the assembly of six subunits (dodecamer or protomer) in the form of a teardrop. The native molecule consists of twelve of such subunits (dodecamer or protomer). Each subunit has a molecular weight of about 250 kDa, and is the functional unit of the native molecule.

Preferably, the Annelid extracellular hemoglobin is chosen from the Polychaete Annelid extracellular hemoglobins and the Oligochaete Annelid extracellular hemoglobins. Preferably, the Annelid extracellular hemoglobin is chosen from extracellular hemoglobins of the Lumbricidae family, extracellular hemoglobins of the Arenicolidae family and extracellular hemoglobins of the Nereididae family. Even more preferentially, the Annelid extracellular hemoglobin is chosen from Lumbricus terrestris extracellular hemoglobin, Arenicola sp extracellular hemoglobin and Nereis sp extracellular hemoglobin More preferentially, according to the invention, the Annelid extracellular hemoglobin is chosen from the Arenicola marina extracellular hemoglobin or Nereis virens extracellular hemoglobin, more preferentially is the Arenicola marina extracellular hemoglobin. Arenicola marina is a polychaete annelid worm living mainly in sand.

According to the invention, the globin protomer of Annelid extracellular hemoglobin constitutes the functional unit of native hemoglobin, as indicated hereinabove.

Finally, the globin chain of Annelid extracellular hemoglobin may in particular be chosen from the globin chains of type Ax and/or Bx of Annelid extracellular hemoglobin.

Annelida extracellular hemoglobin, the globin protomers thereof and/or the globins thereof do not require a cofactor to function, unlike mammalian hemoglobin, in particular human hemoglobin. Finally, Annelid extracellular hemoglobin, the globin protomers thereof and/or the globins thereof do not have blood typing, same make possible to avoidt any problem of immunological or allergic reaction. Annelida extracellular hemoglobin, the globin protomers thereof and/or the globins thereof exhibit intrinsic superoxide dismutase (SOD) activity. Therefore, the intrinsic antioxidant activity does not require any antioxidant to function, unlike the use of mammalian hemoglobin in which antioxidant molecules are contained within the red blood cell and are not bound to hemoglobin.

Annelida extracellular hemoglobin, the globin protomers thereof and/or the globins thereof can be native or recombinant.

Preferably, extracellular hemoglobin is the Arenicola marina hemoglobin or the Nereis virens hemoglobin, more preferentially extracellular Arenicola marina hemoglobin.

Preferably, the molecule is present in a composition in an amount comprised between 0.01% and 10% by weight relative to the total weight of the composition, preferably between 0.05% and 5% by weight, preferably between 0.06% and 2% by weight, preferably between 0.07% and 1% by weight.

Preferably, the use according to the invention significantly reduces intermittent hypoxia of corneal endothelial cells, thus reducing the stress of the endoplasmic reticulum of corneal endothelial cells.

“Significantly reducing intermittent hypoxia of corneal endothelial cells”, means significantly reducing cellular stress induced by nocturnal hypoxia occurring at the pathological endothelium. Such phenomenon occurs at night, during prolonged eyelid closure. The reduction in intermittent hypoxia of corneal endothelial cells can be measured using the hypoxia model described as an example, in particular by measuring the markers MMP2 and/or BAG3.

Preferably, the use according to the invention makes it possible to significantly reduce the expression of MMP2 and/or of BAG3 in the corneal endothelium.

“Significantly reduce the expression of MMP2 and/or BAG3 in the corneal endothelium” means a reduction in the transcription and/or translation of MMP2 and/or BAG3 in the corneal endothelial cells by at least 20%, preferably by at least 30%, preferably by at least 40%, preferably by at least 50%. Preferably, the use of at least one molecule chosen from an Annelid globin, an Annelid globin protomer and an Annelid extracellular hemoglobin according to the invention reduces the transcription and/or translation of MMP2 and/or BAG3 in corneal endothelial cells by at least 20%, preferably by at least 30%, preferably by at least 40%, preferably by at least 50%, compared with control corneal endothelial cells. The controls may be corneal endothelial cells not treated with the molecule, or else corneal endothelial cells before treatment with the molecule. The transcription and/or translation of MMP2 and/or of BAG3 can be measured by any known method of the prior art, and in particular as described as an example.

Preferably, the use according to the invention makes it possible to protect the corneal endothelial cells from hypoxia/reoxygenation stress.

“Protecting corneal endothelial cells from hypoxic stress” means a decrease in the expression of at least one stress marker of the endoplasmic reticulum of corneal endothelial cells, and/or a decrease in the expression of at least one autophagy marker of corneal endothelial cells, preferably BAG3, and/or an increase in the cell viability of corneal endothelial cells, of at least 20%, preferably at least 30%. Preferably, the endoplasmic reticulum stress marker is selected from HSPA5 (BIP), DDIT3 (CHOP) and sXBP1. Preferably, the autophagy marker of corneal endothelial cells is BAG3. Preferably, the use of at least one molecule chosen from an Annelid globin, an Annelid globin protomer and an Annelida extracellular hemoglobin according to the invention decreases the expression of at least one marker of stress of the endoplasmic reticulum of corneal endothelial cells, and/or decreases the expression of at least one marker of autophagy of corneal endothelial cells, preferably BAG3, and/or increases the cellular viability of the corneal endothelial cells, by at least 30%, compared with the control cells, by at least 20%. The controls may be corneal endothelial cells not treated with the molecule, or else corneal endothelial cells before treatment with the molecule. The expression of at least one stress marker of the endoplasmic reticulum of corneal endothelial cells, the expression of at least one autophagy marker of corneal endothelial cells and the cell viability of corneal endothelial cells can be measured according to any method known from the prior art, and in particular as described as an example.

Preferably, the molecule chosen from an Annelid globin, an Annelid globin protomer and an Annelid extracellular hemoglobin according to the invention is formulated in a buffer solution. The resulting solution (i.e. buffer solution comprising the molecule) can be lyophilized to obtain a powder. Preferably, the solution obtained (i.e. buffer solution comprising the molecule) is used as such (liquid form), in a non-lyophilized form.

Typically, the buffer solution creates an adequate saline environment for hemoglobin, the protomers and globins, and thus serves to maintaining the quaternary structure, and hence the functionality of the molecule. The buffer solution is preferably an aqueous solution comprising salts, preferably chloride, sodium, calcium, magnesium and potassium ions, and the pH thereof is comprised between 5 and 9, preferably between 5.5 and 8.5, ideally between 7.4 and 7.7 (which corresponds to the physiological pH of the tear film). The formulation thereof is similar to the formulation of a physiologically injectable liquid. Preferably, the buffer solution also comprises an antioxidant, such as ascorbic acid. Under such conditions, Annelid extracellular hemoglobin, the globin protomers thereof and the globins thereof remain functional.

In the present description, pH is understood to be at room temperature (25° C.) unless otherwise stated. Preferably, the buffer solution is an aqueous solution comprising sodium chloride, calcium chloride, magnesium chloride, potassium chloride, as well as sodium gluconate and sodium acetate, and has a pH between 6.5 and 7.6, preferably 7.1±0.5, preferably about 7.35. More preferably, the buffer solution is an aqueous solution comprising 90 mM NaCl, 23 mM Na-gluconate, 2.5 mM CaCl₂), 27 mM Na-acetate, 1.5 mM MgCl₂, 5 mM KCl, and has a pH of 7.1±0.5. Preferably, the buffer solution has an osmolarity close to the osmolarity of the tear film, i.e. comprised between 270 and 315 mOsm/L, preferably about 300 mOsm/l.

Preferably, the molecule chosen from an Annelid globin, an Annelid globin protomer and an Annelida extracellular hemoglobin according to the invention (formulated or not in a buffer solution) is formulated in a pharmaceutical composition preferably suitable for ocular administration.

“Pharmaceutical composition suitable for ocular administration” refers to any pharmaceutical composition (medication) having a galenic form suitable for ocular administration.

Preferably, the pharmaceutical composition suitable for ocular administration is chosen from eye drops, ophthalmic ointments, ophthalmic gels, conjunctival inserts and therapeutic lenses.

Eye drops are sterile liquid preparations (e.g. solutions, suspensions or emulsions) for the treatment of eye disorders. Same are typically presented in specific multidose bottles of 5 to 10 ml with a dropper tip or in unit doses (ophtadoses). The packaging is typically suitable for the administration of the eye drops, and can provide a maximum volume of one drop of approximately 30 μl. The eye drops generally comprise a solvent, preferably an aqueous solvent. The formulation may comprise an adjuvant, preferably boric acid or a salt thereof, and/or isotonizing agents such as sodium chloride, and/or vitamin C or the derivatives thereof, preferably ascorbic acid. The eye drops may comprise a surfactant, preferably chosen from polysorbates, polyoxyethylenes and tyloxapol. The surfactant improves the solubility of the active ingredient. The eye drops are sterile and isotonic (pH between 6.4 and 7.8).

Ophthalmic ointments have a semi-solid consistency. Same are used to have a longer effect because the active ingredient is kept in contact with the eye for longer. Same are also prescribed in the treatment of eyelid diseases (blepharitis or styes). Among the excipients commonly encountered in ophthalmic ointments, mention may preferably be made of petroleum jelly or liquid paraffin.

Ophthalmic gels are sterile semi-solid preparations intended to be applied to the conjunctiva. Generally, same contain one or a plurality of active principle(s) dissolved in an appropriate excipient. The excipient is typically a hydrophilic polymer that gels in the presence of water, e.g. a carbomer, carbopol or polyacrylic acid.

Conjunctival inserts are devices implanted under the eyelid. An example of such type of insert, based on tropicamide and phenylephrine hydrochloride, is the Mydriasert reference.

Therapeutic lenses are medical devices that maintain binocular vision (e.g. in relation to an eye dressing). Such a lens may be pre-impregnated with the molecule chosen from an Annelid globin, an Annelid globin protomer and an Annelida extracellular hemoglobin according to the invention, either formulated or not formulated in a buffer solution.

A further subject matter of the invention relates to an in vitro model of Fuchs' dystrophy, comprising cells, preferably healthy corneal endothelium cells, and a culture medium comprising cobalt chloride (CoCl2). “Healthy corneal endothelium cells” means cells not affected by Fuchs' dystrophy. Preferably, CoCl2 is present in a concentration ranging from 1 to 15 μm, preferably from 2 μM to 10 μM. Preferably, healthy corneal endothelium cells are the HCEC B4G12 endothelial line.

Cobalt chloride (CoCl2) can be used as an additive in a cell culture medium; when the cell culture medium is with healthy corneal endothelium cells, an in vitro model of Fuchs' dystrophy can be obtained.

The invention is illustrated by the following examples and figures.

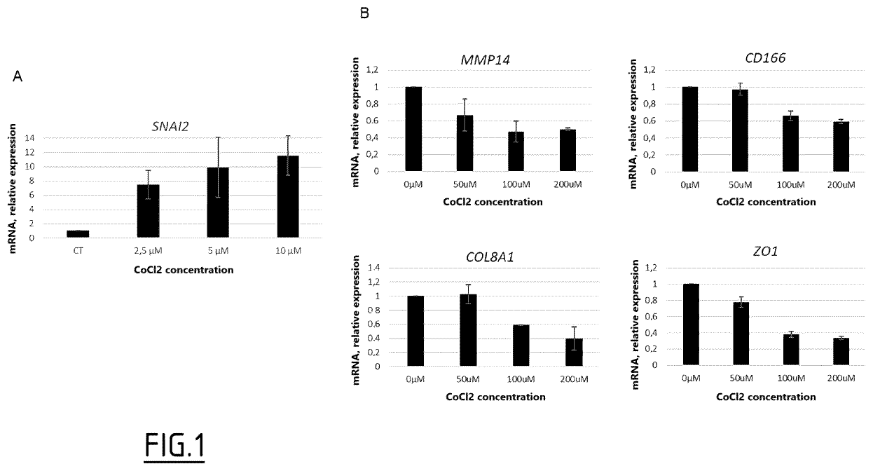

FIG. 1 Cobalt chloride induces a loss of the endothelial phenotype with endothelial-mesenchymal transition on the endothelial line HCEC B4G12.
A: Gene expression, endothelial-mesenchymal transition marker SNA/2, prolonged culture. Prolonged treatment for 7 days with cobalt chloride induces the expression of the endothelial-mesenchymal transition gene SNA/2 in a dose-dependent manner;
B: Gene expression, markers of endothelial differentiation ZO1, COL8A1, MMP14 and CD166, short culture. A short course of treatment (24 h) leads to a dose-dependent decrease in expression of endothelial differentiation markers ZO1, COL8A1, MMP14 and CD166.

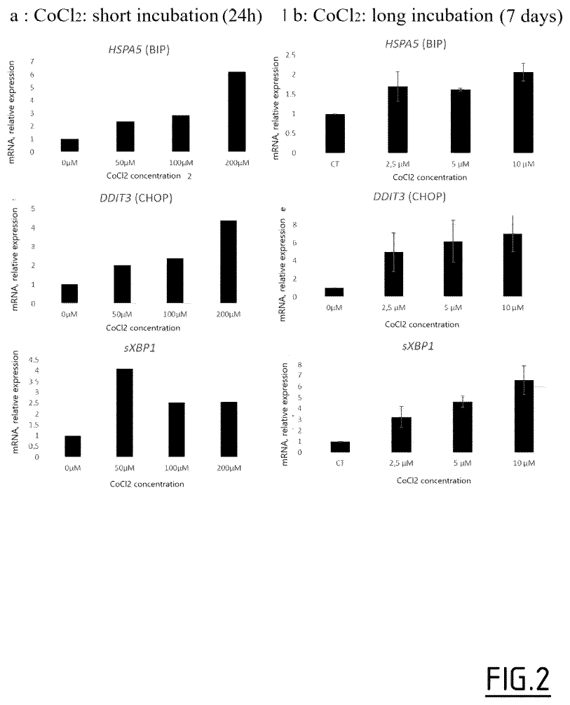

FIG. 2 Induction of gene expression of endoplasmic reticulum stress markers by cobalt chloride (CoCl2).

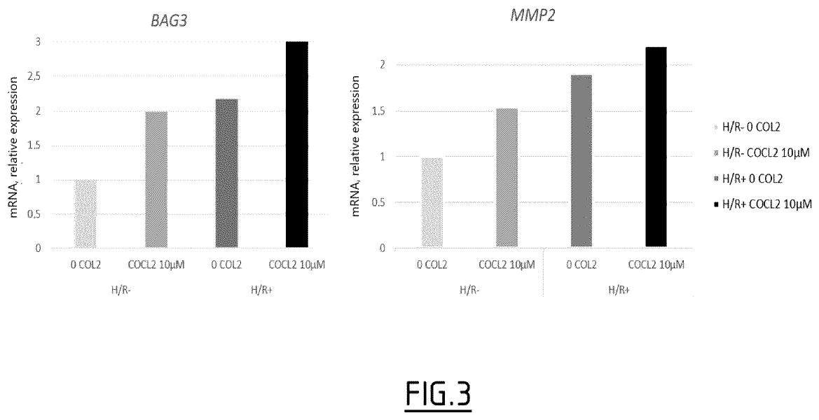

FIG. 3 Induction of gene expression of BAG3 and MMP2 by hypoxia/reoxygenation (H/R) with or without cobalt chloride (CoCl2).

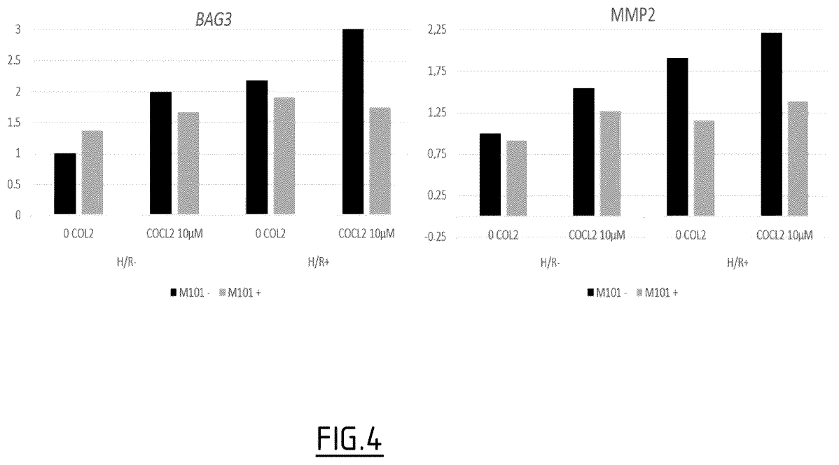

FIG. 4 The Arenicola marina (M101) hemoglobin at 0.5 g/l decreases the effect of hypoxia/reoxygenation (H/R) and cobalt chloride (CoCl2) on the gene expression of BAG3 and MMP2.

### EXAMPLE

### Equipment and Methods:

### Cell Culture:

HCEC-B4G12 (DSZM®, Braunschweig, Germany) is a clonal subpopulation created from the parental cell line HCEC-12 (ACC 646) established from normal corneal endothelium cells of a 91-year-old Caucasian woman transformed with the SV40 genome region. Flasks should be coated with a solution containing 10 μg/mL laminin (L2020, Sigma®, St. Louis, USA) and 10 mg/mL chondroitin sulfate (C4383, Sigma®, St. Louis, USA). The culture medium is composed of Human Endothelial SFM (11111-044 Gibco®, Carlsbad, United States) with 10 ng/mL FGF2 (233-FB-025 Sigma®, Saint-Louis, United States), the cells are cultured at 37° C. with 5% CO2. The culture medium is changed every 48 hours. Cells reach confluence within a week. The cells are passed at confluence after incubation with TripleExpress Trypsin (2604-021 Gibco®, Carlsbad, United States) for 5 minutes at 37° C.

### In Vitro Model of Fuchs' Dystrophy:

Cobalt chloride (CoC2) (C8661, Sigma®, St. Louis, USA) is used as an inducer of proteostasis disorder. A 25 μM stock solution is prepared by diluting 12 mg of cobalt chloride powder in 2 ml of sterile distilled water and stored at 4° C. for one week. Cobalt chloride is then diluted directly in the endothelial culture medium (Human Endothelial SFM+FGF2) to obtain the desired concentrations: 2.5 μM, 5 μM or 10 μM. The media are changed every 48 hours in the presence or in the absence of cobalt chloride with a total culture time of 8 days.

### Cell Senescence Test:

The beta-galactosidase activity of the cells is detected using the senescent cell histochemical staining kit (CS0030, Sigma®, Saint-Louis, USA) according to the supplier's recommendations. HCEC B4G12 cells are cultured for one week with or without cobalt chloride (concentration ranging from 2.5 to 30 μM) in a 24-well plate and then attached with the kit attachment buffer for 7 minutes at room temperature. After rinsing with PBS, the cells are incubated with the staining solution containing X-gal overnight at 37° C. without CO2. After incubation, the cells are observed under a microscope, the cytoplasm of the senescent cells appears blue.

### Hypoxia Model:

A hypoxia incubator (PHCBI®, Bernolsheim, France) was used. Initially, the cells are cultured until confluence in normoxia, about 7 days after seeding, then transferred to hypoxic medium at 1% 02, 5% C02, 94% NO at 37° C. for 12 hours. A hypoxia/reoxygenation (H/R) cycle is then carried out by incubating the cells again in normoxia 21% 02, 5% CO2 at 37° C. after changing the medium. For an analysis of gene expression by RT-qPCR the reoxygenation time is 1 h, for analysis of protein expression by immunofluorescence the reoxygenation time is 3 h.

### Use of M101 in the In Vitro Model:

Arenicola marina hemoglobin (M101) according to the invention is supplied by Hemarina, Morlaix, France. A stock solution of 50 g/l is stored at −80° C. The solution is thawed at room temperature before use and is directly diluted in the culture medium before the hypoxia/reoxygenation test to a concentration ranging from 1 g/l to 0.1 g/l. All the controls are carried out in the presence of the preservation solution at the same volumes as the treated conditions.

### RT-qPCR:

RNA is extracted with the ‘RNAeasy Mini Plus’ kit 74136 (Qiagen®, Hilden, Germany) according to the manufacturer's instructions. The RNA thus isolated was quantified using the Tecan® detection platform (Mannedorf, Switzerland) by performing a spectrophotometric assay. The cDNA of each sample is then obtained by reverse transcription using SuperScript Ill (ThermoFischer®, Waltham, USA) according to the manufacturer's instructions. Each well contains 20 μl of solution with 1 μg of RNA, 10 μl of RT Reaction Mix, 2 μl of enzyme, and a variable supplement of water. Quantitative PCR is then performed for each amplified cDNA sample using the QuantStudio5 aPCR apparatus (ThermoFischer®, Waltham, USA) using the SYBR-Green detection method. The curves are analyzed by the QuantStudio5 software. The primers used for the different genes of interest are described in Table 1. The housekeeping gene, beta-2 microglobulin, used was selected in accordance with the literature data concerning the line. In parallel, different housekeeping genes known in the literature (B2M, GAPDH, PP/A) were tested on different samples of the line. B2M proved to be the least susceptible gene to variations in experimental conditions with a constant detection threshold on the different samples.

TABLE 1

| Genes | | |
|---|---|---|
| of | Primer | Primer |
| interest | Forward | Reverse |
| BAG3 | GGAGATCAAGATCGACCCGC | AGAGGATGGAGTCTCCTTGGG |
| | (SEQ ID NO: 1) | (SEQ ID NO: 2) |
| B2M | ACTGAATTCACCCCCACTGA | CCTCCATGATGCTGCTTACA |
| | (SEQ ID NO: 3) | (SEQ ID NO: 4) |
| CD166 | CCCCAGAGGAATTTTTGTTTTAC | AGCCTGATGTTATCTTTCATCCA |
| | (SEQ ID NO: 5) | (SEQ ID NO: 6) |
| COL8A1 | CCAACTCACCCTTGAAGTCAT | GGCTGGTTCTGTCTCTCTTCAG |
| | (SEQ ID NO: 7) | (SEQ ID NO: 8) |
| DDIT3 | ATGGCAGCTGAGTCATTGCCTTTC | AGAAGCAGGGTCAAGTGGTGAA |
| | (SEQ ID NO: 9) | (SEQ ID NO: 10) |
| HSPA5 | CGAGGAGGAGACAAGAAGG | CACCTTGAACGGCAAGAACT |
| | (SEQ ID NO: 11) | (SEQ ID NO: 12) |
| MMP2 | GTGAAGTATGGGAACGCCGA | AGAAGCCGTACTTGCCATCC |
| | (SEQ ID NO: 13) | (SEQ ID NO: 14) |
| MMP14 | TCCAGCAACTTTATGGGGT | TTCCCGTCACAGATGTTGGG |
| | (SEQ ID NO: 15) | (SEQ ID NO: 16) |
| SNAI2 | TGGTTGCTTCAAGGACACAT | GCAAATGCTCTGTTGCAGTG |
| | (SEQ ID NO: 17) | (SEQ ID NO: 18) |
| sXBP1 | CTGAGTCCGAATCAGGTGCA | ATCCATGGGGAGATGTTCTGG |
| | (SEQ ID NO: 19) | (SEQ ID NO: 20) |
| ZO1 | GGTCAGAGCCTTCTGATCATTC | CATCTACTCCGGAGACTGC |
| | (SEQ ID NO: 21) | (SEQ ID NO: 22) |

### HCEC B4G12 Cell Line Immunofluorescence:

The cells on lamellae are attached with PFA (1040051000 Merck®, Darmstadt, Germany) diluted to 4% for 15 min. The lamellae are incubated with the permeabilization and saturation buffer: 0.5% Tween 20 (85113 ThermoScientific®, Waltham, USA), 1% Triton (437002A VWR® Radnor, USA), 5% albumin fraction V (1.12018.0100 Merck®, Darmstadt, Germany) for 1 h at room temperature and then incubated with primary antibodies for 3 h in the saturation buffer. Dilution of primary antibodies in the saturation buffer is as follows: BAG3 (Rabbit 10599-1-AP Proteintech®) (1/100), MMP2 Rabbit (37150 Abcam®) (1/200), MMP2 Mouse (436000 Invitrogen®) (1/100), alpha-tubulin (MAB1864, Merck®, Darmstadt, Germany) (1/400).

The lamellae are then rinsed with 5 min PBS (×3), then incubated with the mixture of secondary antibodies and DAPI for one hour at room temperature protected from light in the permeabilization and saturation buffer. The dilution of secondary antibodies is as follows: Alexa Fluor 555 goat Anti-rabbit (A21428 ThermoScientific®, Waltham, USA) (1/500) and Alexa Fluor 488 goat Anti-mouse (A11001 Thermo ThermoScientific®, Waltham, USA) (1/500) with DAPI (62248 ThermoScientific®, Waltham, USA) (1/2000) diluted in the saturation buffer. The lamellae are rinsed with PBS 5 min (×3) and then mounted on slides with Permafluor mounting medium (TA-030-FM TermoScientifc®, Waltham, United States). All negative controls are performed in the absence of primary antibodies but in the presence of secondary antibodies. The image acquisition is carried out using the LSM 800 confocal microscope (Zeiss®, Oberkochen, Germany). The images are then analyzed with a Fiji® software. Each primary antibody is tested at least three times under the different experimental conditions.

### Results

### Preliminary Data on Patient Samples (Images not Shown)

In immunofluorescence, an increase in the signal of the endoplasmic reticulum stress marker BIP (or GRP78) was found in patient samples, which corresponds to current literature data placing reticulum stress at the center of the pathophysiology of Fuchs' dystrophy.

On orthogonal projection reconstructions, the inventors were able to demonstrate a loss of the linear distribution of MMP2 within the Descemet membrane in comparison with healthy subjects. On pathological endothelial-Descemet, MMP2 forms intracellular aggregates in the perinuclear region.

A modification of the expression concerning BAG3 is also found. The expression thereof is cytoplasmic and diffuse in immunofluorescence in a healthy subject. In a subject with Fuchs' dystrophy, regional changes in BAG3 expression are observed. BAG3 is found in the perinuclear region at the cells located at the periphery of a pathological endothelial-Descemet (in the drip-free zones), while in the central zone of the drip-rich endothelial-Descemet, the signal of BAG3 is nuclear and forms perinuclear cytoplasmic aggregates co-localizing with MMP2.

An MMP2 and LCIII co-labeling, aggresome markers, on patient samples indicates the presence of aggresomes containing MMP2.

Preliminary data from patient sample analyzes suggest that BAG3 is involved in the formation of aggresomes on pathological endothelial-Descemet wherein MMP2 is found.

Cobalt Chloride Induces a Loss of the Endothelial Phenotype with Endothelial-Mesenchymal Transition on the Endothelial Line HCEC B4G12

During prolonged treatment with cobalt chloride in the culture medium for doses ranging from 2.5 μM to 10 μM, the cells lose the endothelial phenotype thereof with in particular a loss of the contiguous and hexagonal appearance (photos not shown) and the presence of fibroblastic cells with larger and longer cytoplasm.

Similarly, prolonged treatment for 7 days with cobalt chloride induces the expression of the endothelial-mesenchymal transition gene SNA/2 in a dose-dependent manner (FIG. 1A). Beta-galactosidase activity, illustrated by perinuclear cytoplasmic blue staining (photos not shown) of endothelial cells in the presence of cobalt chloride, is also increased, reflecting the phenomenon of cellular senescence induced by the molecule.

Short-term treatment (24 h) results in a dose-dependent decrease in expression of the endothelial differentiation markers ZO1, COL8A1, MMP14 and CD166 (FIG. 1B).

The data suggest that cobalt chloride leads to a loss of endothelial differentiation with endothelial-mesenchymal transition and induces cellular senescence, phenomena observed in vivo in patients with Fuchs' dystrophy.

### Cobalt Chloride Induces Endoplasmic Reticulum Stress on the Endothelial Line HCEC B4G12

Cobalt chloride induces an expression of endoplasmic reticulum markers, HSPA5 (BIP), DDIT3 (CHOP) and sXBP1 (FIG. 2), evaluated by RT-qPCR, either during short incubation for 24 h (FIG. 2a) for doses ranging from 50 to 200 μM, or during prolonged culture for 7 days (FIG. 2b) for doses ranging from 2.5 to 10 μM.

In immunofluorescence, cobalt chloride in prolonged culture induces the formation of MMP2 aggregates at the perinuclear region and along the microtubules. An increase in the BIP signal at the endoplasmic reticulum is also demonstrated in the presence of cobalt chloride (images not shown).

The use of cobalt chloride mimics in vitro endoplasmic reticulum stress and aggresome formation as observed in vivo in patient samples.

### Hypoxia/Reoxygenation Alters the Expression of BAG3 and MMP2 on the HCEC B4G12 Line

Hypoxia/reoxygenation increases BAG3 and MMP2 expression evaluated by RT-qPCR, as much as during an incubation with cobalt chloride for one week. Performing a hypoxia/reoxygenation cycle in the presence of cobalt chloride further increases the expression of BAG3 and MMP2 (FIG. 3).

In immunofluorescence, the BAG3 signal is reinforced at the perinuclear region in the presence of cobalt chloride. The BAG3 signal is found nuclear when a hypoxia/reoxygenation cycle is performed in the presence of cobalt chloride (images not shown).

The nuclear translocation of BAG3, in the same way as on the most pathological zones of patient samples, could indicate an increased susceptibility of endothelial cells to hypoxia/reoxygenation in the presence of pre-existing endoplasmic reticulum stress.

### the Use of M101 in the Presence of Cobalt Chloride or Hypoxia/Reoxygenation Restores the Expression of BAG3 and MMP2

The use of M101 hemoglobin at a concentration of 0.5 g/l decreases gene expression of MMP2 and BAG3 in each treatment condition. The decrease in expression is more pronounced in the hypoxia/reoxygenation condition in the presence of endoplasmic reticulum stress (FIG. 4) generated by the use of cobalt chloride, than in conditions where a hypoxia/reoxygenation cycle is performed alone or where cells are incubated with cobalt in an isolated way.

Immunofluorescence labeling of BAG3 does not show a nuclear signal with M101 (0.5 g/L) in the hypoxia/reoxygenation condition in the presence of cobalt chloride (images not shown).

Such results indicate that the use of M101 on the in vitro model seems to reverse the effect of fluctuations in oxygen levels in culture media and could protect endothelial cells under hypoxic stress conditions.

### CONCLUSION

All the elements presented above allow us to argue that intermittent hypoxia influences the expression of markers involved in the pathophysiology of Fuchs' dystrophy such as BAG3 and MMP2. The effect of hypoxia/reoxygenation is more severe in the presence of pre-existing reticulum stress, which can be observed in patient samples.

Such data suggest that the use of Arenicola marina hemoglobin (M101) in Fuchs' dystrophy, in different possible dosage forms, may limit nocturnal oxygen fluctuations in the corneal endothelium. The use thereof would reduce cellular stress and ultimately cell loss in order to slow the progression of the disease to a stage requiring a cornea transplant, which remains, to date, the only curative treatment available.

### BIBLIOGRAPHIC REFERENCES

1. Bonanno J A. Identity and regulation of ion transport mechanisms in the corneal endothelium. Prog Retin Eye Res. janv 2003; 22(1):69-94.
2. Eghrari A O, Riazuddin S A, Gottsch J D. Overview of the Cornea: Structure, Function, and Development. Prog Mol Biol Transl Sci. 2015; 134:7-23.
3. Elbaz U, Mireskandari K, Tehrani N, Shen C, Khan M S, Williams S, et al. Corneal Endothelial Cell Density in Children: Normative Data From Birth to 5 Years Old. Am J Ophthalmol. janv 2017; 173:134-8.
4. Gain P, Jullienne R, He Z, Aldossary M, Acquart S, Cognasse F, et al. Global Survey of Corneal Transplantation and Eye Banking. JAMA Ophthalmol. fevr 2016; 134(2):167-73.
5. Okumura N, Kitahara M, Okuda H, Hashimoto K, Ueda E, Nakahara M, et al.

Sustained Activation of the Unfolded Protein Response Induces Cell Death in Fuchs' Endothelial Corneal Dystrophy. Invest Ophthalmol Vis Sci. 1 juill 2017; 58(9):3697-707.
6. Xu I, Thériault M, Brunette I, Rochette P J, Proulx S. Matrix metalloproteinases and their inhibitors in Fuchs endothelial corneal dystrophy. Exp Eye Res. 19 févr 2021; 205:108500.
7. Zoega G M, Arnarsson A, Sasaki H, Söderberg P G, Jonasson F. The 7-year cumulative incidence of cornea guttata and morphological changes in the corneal endothelium in the Reykjavik Eye Study. Acta Ophthalmol. mai 2013; 91(3):212-8.
8. Pang K, Lennikov A, Yang M. Hypoxia adaptation in the cornea: Current animal models and underlying mechanisms. Anim Models and Exp Med. déc 2021; 4(4):300-10.

1. A method for treating Fuchs' corneal endothelial dystrophy in a subject, the method comprising:
administering a molecule selected from Annelida globin, Annelida globin protomer, and Annelida extracellular hemoglobin to said subject.

2. The method according to claim 1, wherein the Annelid extracellular hemoglobin is chosen from Polychaete Annelid extracellular hemoglobins and Oligochaete Annelid extracellular hemoglobins.

3. The method according to claim 1, wherein the Annelid extracellular hemoglobin is chosen from extracellular hemoglobins of the family Lumbricidae, extracellular hemoglobins of the family Arenicolidae and extracellular hemoglobins of the family Nereididae, preferably from Lumbricus terrestris extracellular hemoglobin, Arenicola sp extracellular hemoglobin and Nereis sp extracellular hemoglobin.

4. The method according to claim 1, wherein the Annelid extracellular hemoglobin is the Arenicola marina extracellular hemoglobin.

5. The method according to claim 1, wherein the molecule is present in a composition in a content comprised between 0.01% and 10% by weight relative to the total weight of the composition, preferably between 0.05% and 5% by weight, preferably between 0.06% and 2% by weight, preferably between 0.07% and 1% by weight.

6. The method according to claim 1, for significantly reducing intermittent hypoxia of corneal endothelial cells.

7. The method according to claim 1, for reducing the stress of the endoplasmic reticulum of corneal endothelial cells.

8. The method according to claim 1, for significantly reducing the expression of MMP2 and/or BAG3 in the corneal endothelium.

9. The method according to claim 1, for protecting corneal endothelial cells from hypoxic stress.

10. The method according to claim 1, wherein the molecule is formulated in a pharmaceutical composition suitable for ocular administration, preferably chosen from eye drops, ophthalmic ointments, ophthalmic gels, conjunctival inserts and therapeutic lenses.


SEQUENCE LISTING (22 sequences)

SEQ ID NO: 1 - DNA, 20 bp, synthetic construct
  misc_feature 1..20: note = amorce forward BAG3
  source 1..20: mol_type = other DNA
ggagatcaagatcgacccgc

SEQ ID NO: 2 - DNA, 21 bp, synthetic construct
  misc_feature 1..21: note = amorce reverse BAG3
  source 1..21: mol_type = other DNA
agaggatggagtctccttggg

SEQ ID NO: 3 - DNA, 20 bp, synthetic construct
  misc_feature 1..20: note = amorce forward B2M
  source 1..20: mol_type = other DNA
actgaattcacccccactga

SEQ ID NO: 4 - DNA, 20 bp, synthetic construct
  misc_feature 1..20: note = amorce reverse B2M
  source 1..20: mol_type = other DNA
cctccatgatgctgcttaca

SEQ ID NO: 5 - DNA, 23 bp, synthetic construct
  misc_feature 1..23: note = amorce forward CD166
  source 1..23: mol_type = other DNA
ccccagaggaatttttgttttac

SEQ ID NO: 6 - DNA, 23 bp, synthetic construct
  misc_feature 1..23: note = amorce reverse CD166
  source 1..23: mol_type = other DNA
agcctgatgttatctttcatcca

SEQ ID NO: 7 - DNA, 21 bp, synthetic construct
  misc_feature 1..21: note = amorce forward COL8A1
  source 1..21: mol_type = other DNA
ccaactcacccttgaagtcat

SEQ ID NO: 8 - DNA, 21 bp, synthetic construct
  misc_feature 1..21: note = amorce reverse COL8A1
  source 1..21: mol_type = other DNA
ggctggtttctgtctcttcag

SEQ ID NO: 9 - DNA, 24 bp, synthetic construct
  misc_feature 1..24: note = amorce forward DDIT3
  source 1..24: mol_type = other DNA
atggcagctgagtcattgcctttc

SEQ ID NO: 10 - DNA, 24 bp, synthetic construct
  misc_feature 1..24: note = amorce reverse DDIT3
  source 1..24: mol_type = other DNA
agaagcagggtcaagagtggtgaa

SEQ ID NO: 11 - DNA, 19 bp, synthetic construct
  misc_feature 1..19: note = amorce forward HSPA5
  source 1..19: mol_type = other DNA
cgaggaggagacaagaagg

SEQ ID NO: 12 - DNA, 20 bp, synthetic construct
  misc_feature 1..20: note = amorce reverse HSPA5
  source 1..20: mol_type = other DNA
caccttgaacggcaagaact

SEQ ID NO: 13 - DNA, 20 bp, synthetic construct
  misc_feature 1..20: note = amorce forward MMP2
  source 1..20: mol_type = other DNA
gtgaagtatgggaacgccga

SEQ ID NO: 14 - DNA, 20 bp, synthetic construct
  misc_feature 1..20: note = amorce reverse MMP2
  source 1..20: mol_type = other DNA
agaagccgtacttgccatcc

SEQ ID NO: 15 - DNA, 20 bp, synthetic construct
  misc_feature 1..20: note = amorce forward MMP14
  source 1..20: mol_type = other DNA
tccagcaactttatgggggt

SEQ ID NO: 16 - DNA, 20 bp, synthetic construct
  misc_feature 1..20: note = amorce reverse MMP14
  source 1..20: mol_type = other DNA
ttcccgtcacagatgttggg

SEQ ID NO: 17 - DNA, 20 bp, synthetic construct
  misc_feature 1..20: note = amorce forward SNAI2
  source 1..20: mol_type = other DNA
tggttgcttcaaggacacat

SEQ ID NO: 18 - DNA, 20 bp, synthetic construct
  misc_feature 1..20: note = amorce reverse SNAI2
  source 1..20: mol_type = other DNA
gcaaatgctctgttgcagtg

SEQ ID NO: 19 - DNA, 20 bp, synthetic construct
  misc_feature 1..20: note = amorce forward sXBP1
  source 1..20: mol_type = other DNA
ctgagtccgaatcaggtgca

SEQ ID NO: 20 - DNA, 21 bp, synthetic construct
  misc_feature 1..21: note = amorce reverse sXBP1
  source 1..21: mol_type = other DNA
atccatggggagatgttctgg

SEQ ID NO: 21 - DNA, 22 bp, synthetic construct
  misc_feature 1..22: note = amorce forward ZO1
  source 1..22: mol_type = other DNA
ggtcagagccttctgatcattc

SEQ ID NO: 22 - DNA, 21 bp, synthetic construct
  misc_feature 1..21: note = amorce reverse ZO1
  source 1..21: mol_type = other DNA
catctctactccggagactgc